(19) United States
(12) Patent Application Publication
(10) Pub. No.: US 2024/0188563 A1
(43) Pub. Date: Jun. 13, 2024

(54) CATHETER LOCK SOLUTIONS INCORPORATING NITRIC OXIDE RELEASING LIPOSOMES

(71) Applicant: Becton, Dickinson and Company, Franklin Lakes, NJ (US)

(72) Inventor: Brendan Laine, Draper, UT (US)

(21) Appl. No.: 18/078,694

(22) Filed: Dec. 9, 2022

(51) Int. Cl. A01N 33/26; A01N 25/04; A01N 41/12

(52) U.S. Cl. CPC A01N 33/26; A01N 25/04; A01N 41/12

(57) ABSTRACT

Catheter lock solutions contain liposomes which encapsulate a nitric oxide releasing precursor material. The catheter lock solutions release nitric oxide under physiologic conditions to provide antithrombotic and antimicrobial properties. The liposomes may be lyophilized for stable storage to maintain their nitric oxide releasing capacity. A kit for preparing a catheter lock solution includes a quantity of lyophilized liposomes which encapsulate a nitric oxide releasing precursor material and a volume of an aqueous rehydrating solution. A catheter lock solution may be prepared by rehydrating a quantity of lyophilized liposomes which encapsulate a nitric oxide releasing precursor material with an aqueous rehydrating solution in a volume sufficient to fill a catheter.

### BACKGROUND

The disclosed invention relates to catheter lock solutions incorporating nitric oxide releasing liposomes. Nitric oxide releasing precursor materials are incorporated into liposomes, which are then incorporated into catheter lock solutions to release nitric oxide under physiologic conditions and to provide antithrombotic and antimicrobial properties.

Maintaining central venous access is a challenge in patients depending on long-term intravenous therapies, such as chemotherapy, hemodialysis, and home parenteral nutrition. These patients depend on long-term intravenous administration of fluids via a central venous access device, such as a central venous catheter (CVC). Despite preventive hygiene protocols, catheter related blood stream infections (CRBSIs) remain a problem. Thrombosis of CVCs is also a known problem. Use of a catheter lock solution (CLS) is one approach to address these problems.

A catheter lock solution is used to fill the central venous catheter when it is not in use, primarily to prevent clotting. Neutrolin® is an example of a commercially available catheter lock solution made by CorMedix Inc., which contains taurolidine, heparin, and citrate (1.35%, 1000 U/mL, and 3.5%, respectively), compounds which are commonly used to prevent bacterial colonization and thrombosis and to maintain catheter patency. Duralock-C® is another commercially available catheter lock solution made by Medical Components, Inc., which contains a citrate solution of various concentrations.

The catheter lock solution (CLS) may be provided in a volume matching the fill volume of the CVC, typically between about 3 to 5 mL. The CLS is withdrawn prior to use of the catheter. Other ingredients of a typical CLS include sterile water for injection. The pH may be adjusted with citric acid and sodium hydroxide.

State of the art catheter lock solutions, actively commercialized for anticoagulant properties, are primarily citrate- or heparin-based. Heparin is animal-sourced, expensive, and can cause thrombocytopenia. Furthermore, catheter lock solutions based on heparin are not very effective. Citrate-based catheter lock solutions are only effective against thrombus formation at the tip of the catheter because the citrate is unable to diffuse through the catheter shaft.

Experimental technologies reported by Dr. Meyerhoff at the University of Michigan and NOTA Laboratories disclose nitric oxide in catheter lock solutions. However, these are not commercially viable technologies. To make nitric oxide precursor technologies commercially viable, it is necessary to stabilize the release of nitric oxide both during storage and during clinical use due to the nature of the precursor materials. The technologies from Dr. Meyerhoff and NOTA Laboratories rely on either a biodegradable poly(lactic-co-glycolic acid) (PLGA) or a salt-based additive to stabilize this nitric oxide release.

However, salt-based additives only provide stabilization in dissolved form, and they cannot provide a stable solution for reasonable shelf-life storage of the precursor materials, which have inherently short shelf-lives. The PLGA stabilized precursor materials may be more stable, but the biodegradable polymers are known to incite inflammatory and foreign body reactions, especially in vascular applications, in a small portion of the population.

It would be an advancement in the art to provide catheter lock solutions which are able to release nitric oxide and provide antithrombotic and antimicrobial properties to central venous catheters as well as other types of catheters.

The catheter lock solutions disclosed herein which release nitric oxide are not limited to embodiments that solve any disadvantages or that operate only in environments such as those described above. Rather, this background is only provided to illustrate one example technology area where some implementations described herein may be practiced.

### SUMMARY

The present disclosure relates generally to catheter lock solutions comprising liposomes, wherein the liposomes encapsulate a nitric oxide releasing precursor material.

In some embodiments, the liposomes have an outer membrane comprising a lipid material.

In some embodiments, the liposomes have an outer membrane comprising a lipid material and cholesterol, wherein the lipid material and cholesterol are present in an approximately 1:1 molar ratio. As used herein, “an approximately 1:1 molar ratio” includes molar ratios ranging from 0.9:1 to 1:0.9. In some embodiments, the lipid material and cholesterol are present in a molar ratio ranging from 0.8:1 to 1:0.8. In some embodiments, the lipid material and cholesterol are present in a molar ratio ranging from 0.7:1 to 1:0.7. In some embodiments, the lipid material and cholesterol are present in a molar ratio ranging from 0.6:1 to 1:0.6. In some embodiments, the lipid material and cholesterol are present in a molar ratio ranging from 0.5:1 to 1:0.5.

In some embodiments, the lipid material comprises a phosphocholine material. In some embodiments, the phosphocholine material comprises dipalmitolylphosphatidylcholine (DPPC).

In some embodiments, the liposomes have an outer membrane comprising a lipid material selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

In some embodiments, the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

In some embodiments, the nitric oxide releasing precursor material comprises N-diazeniumdiolate-modified spermine (SPER/NO).

In some embodiments, the liposomes have an outer membrane comprising a lipid material and cholesterol, selected from wherein the lipid material is dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

In some embodiments, the liposomes have an outer membrane comprising a lipid material and cholesterol, wherein the lipid material and cholesterol are present in an approximately 1:1 molar ratio, the lipid material comprises dipalmitolylphosphatidylcholine (DPPC), and the nitric oxide releasing precursor material comprises N-diazeniumdiolate-modified spermine (SPER/NO).

In some embodiments, the nitric oxide releasing precursor material has a nitric oxide releasing capacity, and wherein the nitric oxide releasing precursor retains at least 80% of its nitric oxide releasing capacity after three months of storage.

The present disclosure further relates to a method for preparing a catheter lock solution comprising liposomes, wherein the liposomes encapsulate a nitric oxide releasing precursor material.

In some embodiments, the method for preparing a catheter lock solution includes obtaining a quantity of lyophilized liposomes which encapsulate a nitric oxide releasing precursor material. In some embodiments, the method for a preparing a catheter lock solution includes rehydrating the lyophilized liposomes to form the catheter lock solution.

In some embodiments of the method for preparing a catheter lock solution, the lyophilized liposomes have an outer membrane comprising a lipid material, wherein the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

In some embodiments of the method for preparing a catheter lock solution, the lyophilized liposomes have an outer membrane comprising a lipid material and cholesterol.

In some embodiments of the method for preparing a catheter lock solution, the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

The present disclosure further relates to a kit for preparing a catheter lock solution comprising liposomes, wherein the liposomes encapsulate a nitric oxide releasing precursor material.

In some embodiments, the kit includes a quantity of lyophilized liposomes which encapsulate a nitric oxide releasing precursor material. In some embodiments, the kit includes a quantity of an aqueous rehydrating solution.

In some embodiments, the lyophilized liposomes have an outer membrane comprising a lipid material selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

In some embodiments, the lyophilized liposomes have an outer membrane comprising a lipid material and cholesterol.

In some embodiments, the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

The present disclosure further relates to a method of preparing liposomes which encapsulate a nitric oxide releasing precursor material.

In some embodiments, the method for preparing liposomes which encapsulate a nitric oxide releasing precursor material includes combining a lipid material and cholesterol in an organic solvent to form an organic phase. In some embodiments the lipid material and cholesterol are present in an approximately 1:1 molar ratio. In some embodiments, the amount of lipid material and cholesterol dissolved in the organic solvent may range from about 0.5 mg/mL to 20 mg/mL.

In some embodiments, the method for preparing liposomes which encapsulate a nitric oxide releasing precursor material includes adding an aqueous solution comprising a nitric oxide releasing precursor material to the organic phase. In some embodiments, the nitric oxide releasing precursor material has a concentration in the range from about 10 mM to 100 mM in the aqueous solution.

In some embodiments, the pH of the aqueous solution is adjusted to be a basic pH. In some embodiments, a sodium hydroxide solution is used to adjust the aqueous solution pH. In some embodiments, the basic pH may be a pH in the range from about pH 7.1 to pH 13. In some embodiments, the sodium hydroxide solution has a concentration in the range from about 10 to 200 mM. In some embodiments, the sodium hydroxide solution has a concentration of approximately 50 mM.

In some embodiments, sonication is used to promote liposome formation after adding the aqueous solution to the organic phase. In some embodiments, sonication is performed at a temperature in the range from 20° C. to 60° C. In some embodiments, sonication is performed for a time period ranging from about 2 min to 10 min. In some embodiments, sonication is performed at a temperature of 45° C. for a time period of about 4 minutes.

In some embodiments, the method for preparing liposomes which encapsulate a nitric oxide releasing precursor material includes evaporating the organic solvent, causing liposomes to form and encapsulate the nitric oxide releasing precursor material.

In some embodiments of the method for preparing liposomes, the organic solvent is selected from chloroform, methanol, diethyl ether, isopropyl ether, cyclohexane, ethanol, and mixtures thereof.

In some embodiments of the method for preparing liposomes, the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

In some embodiments of the method for preparing liposomes, the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

In some embodiments of the method for preparing liposomes, the lipid material comprises dipalmitolylphosphatidylcholine (DPPC) and the nitric oxide releasing precursor material comprises N-diazeniumdiolate-modified spermine (SPER/NO).

In some embodiments, the method for preparing liposomes which encapsulate a nitric oxide releasing precursor material includes lyophilizing the liposomes.

In some embodiments of the method for preparing liposomes, the nitric oxide releasing precursor material has a nitric oxide releasing capacity, and wherein the nitric oxide releasing precursor retains at least 80% of its nitric oxide releasing capacity after three months of storage.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a representative illustration of a liposome containing a nitric oxide precursor material.

FIG. 2A shows nitric oxide release profiles of various nitric oxide releasing precursor materials encapsulated in liposomes for the first three hours in 10 mM PBS (pH 7.4, 37° C.). Release profile (a) is from N-diazeniumdiolate-modified dipropylenetriamine (DPTA/NO). Release profile (b) is from a 50:50 molar mixture of DPTA/NO and N-diazeniumdiolate-modified spermine (SPER/NO). Release profile (c) is from SPER/NO.

FIG. 2B shows a nitric oxide release profile (d) of free SPER/NO for the first three hours in 10 mM PBS (pH 7.4, 37° C.).

FIG. 3 shows the nitric oxide release profiles from liposomes encapsulating two different nitric oxide precursor materials, N-diazeniumdiolate-modified diethylenetriamine (DETA/NO) and N-diazeniumdiolate-modified N-propyl-1,3-propanediamine (PAPA/NO) in 10 mM PBS (pH 7.4, 37° C.) over the first 72 hours.

FIG. 4 shows platelet aggregation rate during dialysis with (NO (+)) and without (NO (−)) nitric oxide precursor material in the dialysis fluid.

FIG. 5 is a two-dimensional representation of clot area (cm²) on S-nitroso-N-acetyl penicillamine (SNAP)/CarboSil and control catheters in rabbit veins for 7 hours.

### DESCRIPTION OF EMBODIMENTS

The disclosure relates to catheter lock solutions containing nitric oxide releasing liposomes. One or more nitric oxide releasing precursor materials are incorporated into liposomes, which are then incorporated into catheter lock solutions. The nitric oxide releasing precursor materials release nitric oxide to provide antithrombotic and antimicrobial properties. Nitric oxide is released in gaseous form from the liposomes at physiologically relevant levels and permeates the indwelling catheter, preventing platelet activation and subsequent thrombus formation.

Nitric oxide is a well-documented antithrombotic and antimicrobial agent. It is produced naturally by vascular endothelium to prevent platelet activation and as a natural response to foreign bodies and infections. Nitric oxide precursor materials, however, degrade rapidly in aqueous environments, causing rapid release of the nitric oxide and a short lifetime of efficacy.

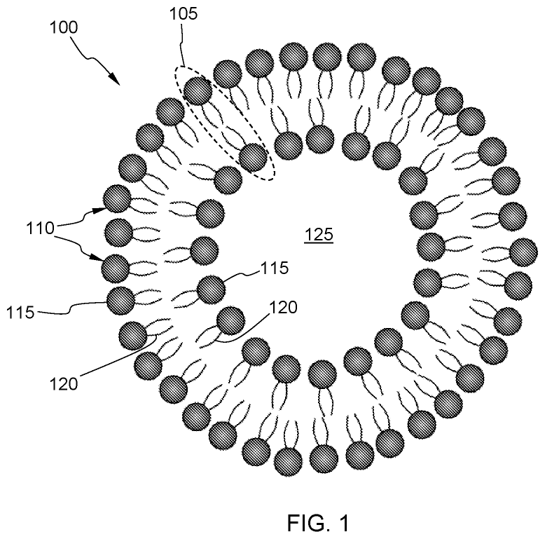

A representative illustration of a liposome 100 is shown in FIG. 1. A liposome is a small spherical structure having a lipid bilayer 105. The lipid bilayer is formed from two layers of lipid molecules 110, oriented such that the hydrophilic portions 115 of the lipid molecules are on the outside surfaces of the lipid bilayer and the hydrophobic portions 120 of the lipid molecules are on the inside of the lipid bilayer. A liposome has an aqueous solution core 125 surrounded by the lipid bilayer. As disclosed herein, the aqueous solution core 125 contains one or more nitric oxide releasing precursor materials.

Liposome encapsulation of nitric oxide releasing precursor materials enables protection from external environments and tunability of the internal aqueous solution core 125 where the nitric oxide releasing precursor materials are dissolved. This stabilizes degradation during storage and enables modulation of the release of nitric oxide in vivo. Liposomes are well suited for this, as opposed to other controlled release mechanisms, because of their well proven in vivo biocompatibility. Liposomes were the first nanoscale drug to be approved for clinical use in 1995. Clinically, liposomes are used as carriers for biologically active molecules and are nontoxic to humans. Zylberberg C, Matosevic S. Pharmaceutical liposomal drug delivery: a review of new delivery systems and a look at the regulatory landscape. Drug Deliv. 2016 Nov.; 23(9):3319-3329. doi: 10.1080/10717544.2016.1177136. Epub 2016 May 5. PMID: 27145899. Other potential controlled release mechanisms are based on biodegradable polymer systems, which are known to incite a foreign body response in vascular applications for a small portion of the population.

Since liposomes are made from lipid-based materials, they provide a natural barrier to aqueous diffusion. Thus, by encapsulating nitric oxide precursor materials within the liposomes, the degradation mechanism is slowed. Furthermore, the lipids provide a barrier to diffusion of the nitric oxide, which stabilizes its release for a longer time period.

The internal environment of the liposomes within the aqueous solution core can be adjusted to further stabilize the nitric oxide precursor materials. For instance, the pH of the aqueous solution core may be adjusted. The pH may be controlled with a buffer. One or more stabilizer chemistries may be used depending upon the choice of nitric oxide precursor material.

Liposomes can be lyophilized and stored in a dehydrated state for long periods of time. This improves the commercialization potential for the disclosed liposomes containing nitric oxide precursor materials. Existing nitric oxide releasing technologies in polymer coatings or solution applications are based on nitric oxide precursor materials directly incorporated into polymers or the solutions. As noted, these precursor materials suffer from rapid degradation under non-refrigerated storage conditions. The nitric oxide is released rapidly with minimal control during in vivo implementation as it degrades immediately in aqueous environments. In contrast, encapsulating nitric oxide releasing precursor materials in liposomes as disclosed herein enables protection from external environments. This stabilizes degradation during storage and enables modulation of the release of nitric oxide in vivo.

Nitric oxide is gaseous so it can permeate the catheter wall to prevent thrombus along the catheter surface as well as preventing occlusions at the tip. Elevating local nitric oxide concentrations may also enhance vasodilation in the tissue surrounding the device, reducing frictional irritation to the vasculature. Furthermore, unlike citrate and heparin, nitric oxide has antimicrobial properties to fight device related infections.

One disclosed method of preparing liposomes which encapsulate a nitric oxide releasing precursor material includes combining a lipid material and cholesterol in an organic solvent for form an organic phase. The cholesterol stabilizes the liposomes mechanically. In some embodiments, the lipid material and cholesterol are present in an approximately 1:1 molar ratio. The method further includes adding an aqueous solution comprising a nitric oxide releasing precursor material to the organic phase. In some embodiments, the method further includes evaporating the organic solvent, causing liposomes to form and encapsulate the nitric oxide releasing precursor material.

In some embodiments, the liposomes which encapsulate a nitric oxide releasing precursor material may be lyophilized to stabilize the liposomes as a dehydrated powder for an extended time period.

Furthermore, lyophilized liposomes may be packed as a kit for preparing a catheter lock solution. The kit may contain a quantity of lyophilized liposomes which encapsulate a nitric oxide releasing precursor material and a quantity of an aqueous rehydrating solution. The aqueous rehydrating solution may be provided in a volume sufficient to fill a catheter. In some embodiments, the catheter may include central venous catheter, and the aqueous rehydrating solution may be provided in the volume sufficient to fill the central venous catheter. In some embodiments, the catheter may include a peripheral intravenous catheter, an arterial catheter, a midline catheter, a peripherally-inserted central catheter, or another suitable type of catheter. Using the disclosed kit, a catheter lock solution containing liposomes which encapsulate a nitric oxide releasing precursor material may be prepared as needed prior to administration by health care persons.

Other features and advantages of the disclosed invention are apparent from the different examples that follow. The examples below illustrate different aspects and embodiments of the present invention and how to make and practice them. The examples do not limit the claimed invention. Although methods and materials similar or equivalent to those described herein can be used in the practice of the present invention, suitable methods and materials are described below. Based on the present disclosure the skilled artisan can identify and employ other components and methodology useful for practicing the present invention.

Example 1—Preparation of liposomes which encapsulate a nitric oxide releasing precursor material.

A lipid material and cholesterol are dissolved in an organic solvent to form an organic phase. The amount of lipid material and cholesterol dissolved in the organic solvent ranges from about 0.5 mg/mL to 20 mg/mL. In this example, the lipid material comprises dipalmitoylphosphatidylcholine (DPPC) and the organic solvent is a mixture of diethyl ether and chloroform. Other organic solvents, such as methanol, diethyl ether, isopropyl ether, cyclohexane, ethanol, and mixtures thereof may also be used. The DPPC and cholesterol are present in an approximate 1:1 molar ratio. An aqueous solution comprising a nitric oxide releasing precursor material is added to and mixed with the organic phase. The nitric oxide releasing precursor material has a concentration in the range of about 10 to 100 mM in the aqueous solution. In this example, the nitric oxide releasing precursor material is N-diazeniumdiolate-modified spermine (SPER/NO). The SPER/NO has a concentration of 14 mM in the aqueous solution. Sterile water is used to prepare the aqueous solution. The pH of the aqueous solution may be adjusted to be a basic pH with a sodium hydroxide solution. The basic pH may be a pH in the range from about pH 7.1 to pH 13. The sodium hydroxide solution may have a concentration in the range from about 10 to 200 mM. The sodium hydroxide solution may have a concentration of approximately 50 mM. Sonication is used to promote liposome formation after adding the aqueous solution to the organic phase. Sonication may be performed at a temperature in the range from 20° C. to 60° C. Sonication may be performed for a time period ranging from about 2 min to 10 min. In this example, sonication is performed at a temperature of 45° C. for a time period of about 4 minutes. The liposomes are generated spontaneously from the lipid material and cholesterol, which creates a bilayer sphere from the energetically favorable interaction of the lipophilic molecules in an aqueous system and encapsulate the aqueous solution containing the nitric oxide releasing precursor material. The organic solvent is evaporated.

Many different lipid materials may be used to prepare the liposome outer membrane as disclosed herein. Non-limiting examples of lipid materials which may be used to prepare a liposome include dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

In some non-limiting embodiments, one lipid is combined with cholesterol in a 1:1 molar ratio to form a lipid bilayer membrane of the liposome.

Many different nitric oxide precursor materials may be used to prepare the liposomes as disclosed herein. Non-limiting examples of nitric oxide precursors materials include N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

Example 2—Preparation of a catheter lock solution from lyophilized liposomes encapsulating a nitric oxide precursor material.

A quantity of lyophilized liposomes which encapsulate a nitric oxide releasing precursor material is obtained. The liposomes are rehydrated with a volume of an aqueous solution sufficient to fill a catheter. The solution of rehydrated liposomes is used as a catheter lock solution to fill a catheter.

### Proof of Concept Data

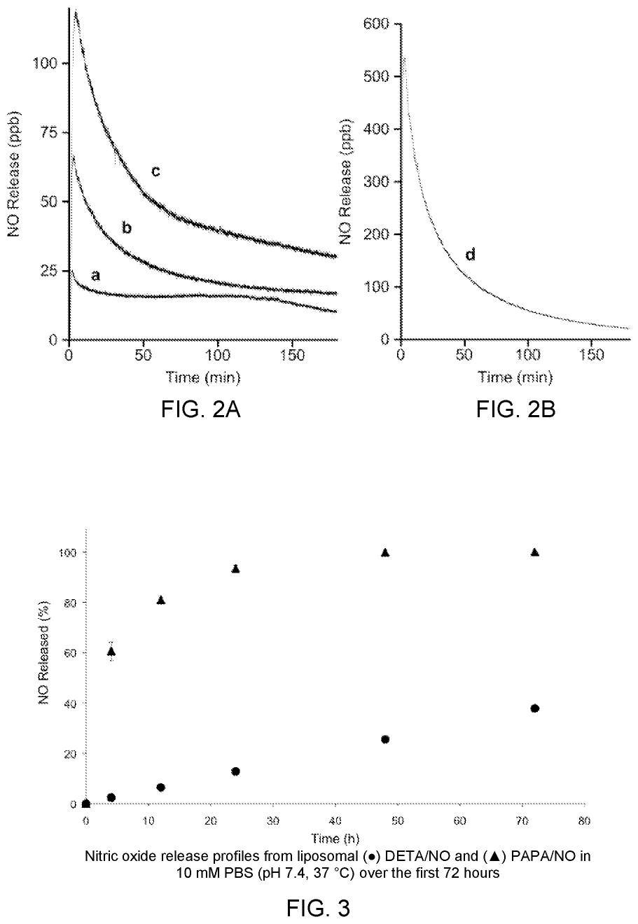

FIG. 2A shows nitric oxide release profiles of various nitric oxide releasing precursor materials encapsulated in liposomes for the first three hours in 10 mM PBS (pH 7.4, 37° C.). Release profile (a) is from N-diazeniumdiolate-modified dipropylenetriamine (DPTA/NO). Release profile (b) is from a 50:50 molar mixture of DPTA/NO and N-diazeniumdiolate-modified spermine (SPER/NO). Release profile (c) is from SPER/NO. FIG. 2B shows a nitric oxide release profile of free SPER/NO for the first three hours in 10 mM PBS (pH 7.4, 37° C.). The liposomes encapsulating nitric oxide releasing precursor materials used in profiles (a), (b), and (c), were synthesized by dissolving the SPER/NO and/or DPTA/NO in 50 mM NaOH. This solution was added to dipalmitoylphosphatidylcholine (DPPC)/cholesterol organic mixture and briefly sonicated to form an emulsion. The organic phase was evaporated to yield liposomes have an approximate diameter of 275 nm.

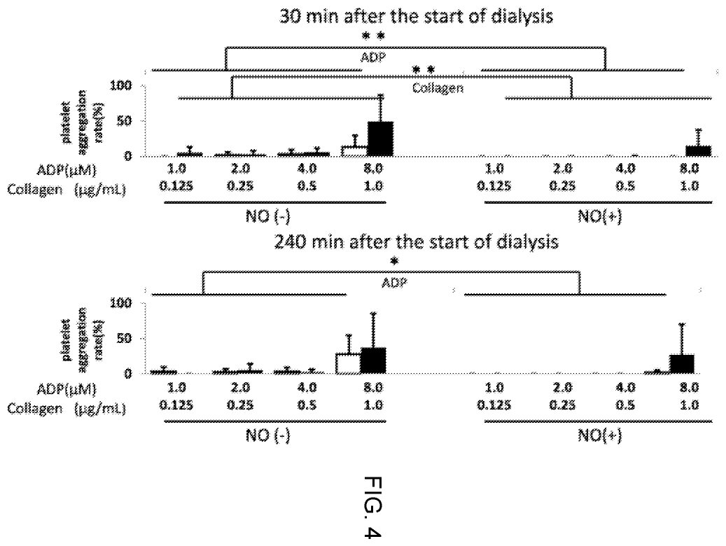

FIG. 3 shows the nitric oxide release profiles from liposomes encapsulating two different nitric oxide precursor materials in 10 mM PBS (pH 7.4, 37° C.) over the first 72 hours. Liposome (●) contained N-diazeniumdiolate-modified diethylenetriamine (DETA/NO) as the nitric oxide precursor material. Liposome (▴) contained N-diazeniumdiolate-modified N-propyl-1,3-propanediamine (PAPA/NO) as the nitric oxide precursor material. The liposomes were prepared by dissolving a 1:1 molar ratio of lipid to cholesterol in an organic solvent mixture of diethyl ether and chloroform. The nitric oxide releasing precursor materials were dissolved in 10 mM NaOH and mixed with the organic solvent mixture and sonicated. The organic solvent was evaporated to yield the liposomes.

The data presented in FIGS. 2A, 2B, and 3 are taken from Suchyta, Dakota. Synthesis And Anticancer Action Of Nitric Oxide-releasing Liposomes. 2017. https://doi.org/10.17615/jqpf-3y 15, which is incorporated by reference.

The data shown in FIGS. 2A, 2B, and 3 indicate a significant improvement in the stabilization of nitric oxide release from precursor materials that are dissolved in liposomes as opposed to precursor materials freely dissolved in solution. Furthermore, the nitric oxide release is stabilized to less than 50% release at 3 days, indicating significant viability for duration of a lock solution's implementation in a clinical setting.

From the data shown in FIGS. 2A, 2B, and 3, liposomes are able to stabilize nitric oxide release in clinically relevant conditions. Liposomes containing nitric oxide releasing precursor materials have also been shown to retain 80% of nitric oxide after 3 months of storage. Additional procedures, such as lyophilization, can also be used to extend shelf-life storage in the dry state followed by resuspension for clinical administration.

The data presented in FIG. 4 is taken from Urabe, S, Kokubo, K., Tsukao, H. et al. Suppression of platelet reactivity during dialysis by addition of a nitric oxide donor to the dialysis fluid. Ren Replace Ther 6. 37 (2020). FIG. 4 shows platelet aggregation rate during dialysis with (NO (+)) and without (NO (−)) nitric oxide precursor material in the dialysis fluid. The white bars show the increase in the platelet aggregation activity in response to the addition of ADP, and the black bars show the increase in the platelet aggregation activity in response to the addition of collagen. At the start of dialysis, there was no difference in the platelet aggregation activity between the NO (−) and NO (+) conditions. However, at 30 min after the start of dialysis, the platelet aggregation activity in the NO (+) condition was significantly decreased as compared with that in the NO (−) condition (P<0.05).

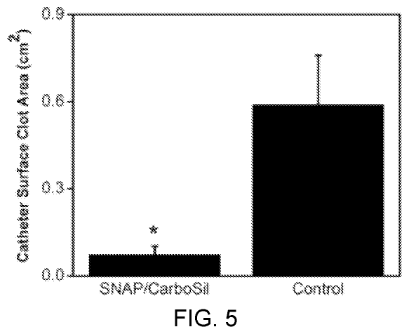

The thromboresistant activity of a nitric oxide releasing precursor material, S-nitroso-N-acetylpenicillamine (SNAP)-doped CarboSil polymer was evaluated. CarboSil is a tri-copolymer of polyurethane, poly(dimethylsiloxane) and polycarbonate. One SNAP/CarboSil catheter and one control catheter (non-SNAP) were placed into the external jugular veins of rabbits for 7 hours. After 7 hours of implantation, the catheters were carefully removed from the rabbit veins, leaving the thrombus formation intact on the surface. Digital images of the catheter surfaces were taken, and the two-dimensional representation of the thrombus area was quantified. FIG. 5 is a two-dimensional representation of clot area (cm²) on SNAP/CarboSil and control catheters in rabbit veins for 7 hours. FIG. 5 shows the thrombus formation is significantly reduced for the SNAP/CarboSil catheters with nitric oxide release when compared to the controls. This demonstrates that localized nitric oxide release from the catheter surfaces can significantly reduce platelet activation and thrombus formation. The data presented in FIG. 5 and Table 1, below, are taken from Yaqi Wo, S-Nitroso-N-acetylpenicillamine (SNAP)-Based Antithrombotic and Antimicrobial Polymers for Biomedical Applications: Nitric Oxide (NO) to the Rescue, PhD Dissertation, University of Michigan, 2017.

Extracorporeal blood circuits are used for several different types of medical procedures, such as hemodialysis, cardiac bypass surgery, and extracorporeal membrane oxygenation (ECMO). Due to the large surfaces area that is in contact with blood in extracorporeal circulation (ECC), the loss of platelet count and platelet functionality are significant. Researchers have developed nitric oxide releasing or nitric oxide generating polymeric tubing to prevent platelet activation and consumption during the ECC procedure. Table 1 is a summary of nitric oxide releasing and nitric oxide generating coatings to increase hemocompatibility of extracorporeal circuits. It reflects the percentage of viable platelets left in a closed blood loop with a nitric oxide-doped catheter. More platelet activation caused by the device will lead to less platelets in the blood flow at the end of the test time.

TABLE 1

| | NO releasing | NO level | |
|---|---|---|---|
| ECC coating | material, | (×10⁻¹⁰ mol | Platelet count after 4 h |
| material | additives, etc. | min⁻¹ cm²) | (% of initial) |
| Tygon | N- | 4.1 | ca. 85 ± 15% (NO) |
| tubings with | diazeniumdiolated | | vs. 58 ± 5% (PU) |
| Carbonthane | FS fillers | | |
| 3573A coating | | | |
| Silicone rubber | N- | >4.0 at | 86 ± 24% (NO) |
| tubings | diazeniumdiolated | 23° C.; | vs. 65 ± 10% (SR) |
| | PDMS | >10.0 at | |
| | | 37° C. | |
| Tygon tubings | DBHD/NONOate, | 12.5 | 79 ± 7% (NO) |
| with PVC/DOS | borate | | vs. 58 ± 7% (PVC/ |
| coating | | | DOS) |
| Tygon tubings | DBHD/NONOate, | >20 | 79 ± 11% (NO) |
| with PVC/DOS | PLGA | | vs. 54 ± 6% (PVC/ |
| coating | | | DOS) |
| Tygon tubings | DBHD/NONOate, | 6 | 97 ± 10% (NO) |
| with Elasteon- | PLGA | | vs. 58 ± 3% (E2As) |
| E2As coating | | | |
| Tygon tubings | DBHD/NONOate, | 6.5 | ca. 90% (NO/ |
| with Elasteon- | PLGA, argatroban | | argatroban) vs. |
| E2As coating | | | 58 ± 3% (E2As) |
| Tygon tubings | SNAP | 2 | 100 ± 7% (NO) |
| with Elasteon- | | | vs. 60 ± 6% (E2As) |
| E2As coating | | | |
| Tygon tubings | endogenous | >10 (in | ca. 90% (10 wt % Cu⁰ |
| with Tecophilic | RSNO and/or | presence of | and SNAP) vs. 75% |
| SP-60D-60 | infused SNAP | 1 μM GSNO, | (10 wt % Cu⁰ only) |
| coating | | 30 μM GSH, | vs. 50% (SNAP only) |
| containing | | 5 μM EDTA) | |
| 10 wt % Cu⁰ | | | |
| nanoparticle | | | |

### EMBODIMENTS

Various embodiments are listed below. It will be understood that the embodiments listed below may be combined with all aspects and other embodiments in accordance with the scope of the invention.

Embodiment 1. An aqueous catheter lock solution comprising liposomes, wherein the liposomes encapsulate a nitric oxide releasing precursor material.

Embodiment 2. The catheter lock solution according to embodiment 1, wherein the liposomes have an outer membrane comprising a lipid material.

Embodiment 3. The catheter lock solution according to embodiments 1 or 2, wherein the liposomes have an outer membrane comprising a lipid material and cholesterol.

Embodiment 4. The catheter lock solution according to embodiment 3, wherein the lipid material and cholesterol are present in an approximately 1:1 molar ratio.

Embodiment 5. The catheter lock solution according to any of embodiments 2 through 4, wherein the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

Embodiment 6. The catheter lock solution according to any of embodiments 2 through 4, wherein the lipid material comprises a phosphocholine material.

Embodiment 7. The catheter lock solution according to embodiment 6, wherein the phosphocholine material comprises dipalmitolylphosphatidylcholine (DPPC).

Embodiment 8. The catheter lock solution according to any preceding embodiment, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

Embodiment 9. The catheter lock solution according to embodiments 1 through 7, wherein the nitric oxide releasing precursor material comprises N-diazeniumdiolate-modified spermine (SPER/NO).

Embodiment 10. The catheter lock solution according to embodiment 1, wherein the liposomes have an outer membrane comprising a lipid material and cholesterol, wherein the lipid material is selected from dipalmitoylphosphatidylcholine (DPPC), dimyristoylphosphatidyl-choline (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

Embodiment 11. The catheter lock solution according to embodiment 10, wherein the lipid material and cholesterol are present in an approximately 1:1 molar ratio, the lipid material comprises dipalmitolylphosphatidylcholine (DPPC), and the nitric oxide releasing precursor material comprises N-diazeniumdiolate-modified spermine (SPER/NO).

Embodiment 12. The catheter lock solution according to any preceding embodiment, wherein the nitric oxide releasing precursor material has a nitric oxide releasing capacity, and wherein the nitric oxide releasing precursor retains at least 80% of its nitric oxide releasing capacity after three months of storage.

Embodiment 13. A method for preparing the catheter lock solution according to embodiment 1, comprising: obtaining a quantity of lyophilized liposomes which encapsulate a nitric oxide releasing precursor material; and rehydrating the lyophilized liposomes to form the catheter lock solution.

Embodiment 14. The method according to embodiment 13, wherein the lyophilized liposomes have an outer membrane comprising a lipid material and cholesterol, wherein the lipid material is selected from dipalmitoylphosphatidylcholine (DPPC), dimyristoylphosphatidyl-choline (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

Embodiment 15. A kit for preparing the catheter lock solution according to embodiment 1, comprising: a quantity of lyophilized liposomes which encapsulate a nitric oxide releasing precursor material; and a quantity of an aqueous rehydrating solution.

Embodiment 16. The kit according to embodiment 15, wherein the lyophilized liposomes have an outer membrane comprising a lipid material and cholesterol, wherein the lipid material is selected from dipalmitoylphosphatidylcholine (DPPC), dimyristoylphosphatidyl-choline (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

Embodiment 17. The catheter lock solution according to an preceding embodiment, wherein the nitric oxide releasing precursor material is dissolved in an aqueous solution having a basic pH in the range from about 7.1 to about 13.

Embodiment 18. A method of preparing liposomes which encapsulate a nitric oxide releasing precursor material, comprising: combining a lipid material and cholesterol in an organic solvent for form an organic phase, wherein the lipid material and cholesterol are present in an approximately 1:1 molar ratio; adding an aqueous solution comprising a nitric oxide releasing precursor material to the organic phase; and evaporating the organic solvent, causing liposomes to form and encapsulate the nitric oxide releasing precursor material.

Embodiment 19. The method of preparing liposomes according to embodiment 18, wherein the organic solvent is selected from chloroform, methanol, diethyl ether, methanol, isopropyl ether, cyclohexane, ethanol, and mixtures thereof.

Embodiment 20. The method of preparing liposomes according to embodiments 18 or 19, wherein the lipid material comprises dipalmitolylphosphatidylcholine (DPPC) and the nitric oxide releasing precursor material comprises N-diazeniumdiolate-modified spermine (SPER/NO).

Embodiment 21. The method of preparing liposomes according to an of embodiments 18 through 20, wherein the aqueous solution has a basic pH in the range from about 7.1 to about 13.

Embodiment 22. The method of preparing liposomes according to embodiment 21, wherein the aqueous solution pH is adjusted with sodium hydroxide.

Embodiment 23. The method of preparing liposomes according to any of embodiments 18 through 22, further comprising the step of lyophilizing the liposomes.

Embodiment 24. The method of preparing liposomes according to any of embodiments 18 through 23, wherein the nitric oxide releasing precursor material has a nitric oxide releasing capacity, and wherein the nitric oxide releasing precursor retains at least 80% of its nitric oxide releasing capacity after three months of storage.

The disclosed catheter lock solutions containing nitric oxide releasing liposomes represent an improvement over heparin and citrate-based lock solutions by leveraging the active ingredient, nitric oxide, that diffuses via gas-phase through the walls of the catheter, to provide antithrombotic activity to the entire surface of the catheter device in addition to preventing occlusions. Furthermore, unlike heparin and citrate, nitric oxide has antimicrobial properties to help fight device-associated infections. Lastly, nitric oxide is produced naturally in the blood stream by vascular endothelial cells. This nitric oxide releasing liposomes release nitric oxide at physiologically relevant concentrations. Therefore, the nitric oxide active agent is inherently biocompatible.

The disclosed catheter lock solutions containing nitric oxide releasing liposomes represents an improvement with respect to nitric oxide-based catheter coating technologies. In the case of central venous catheters, these coatings need to be effective sometimes in excess of 30 days. This is a significant challenge for nitric oxide-based coating technologies, as they generally degrade rapidly and on the order of a few days. A lock solution-based technology improves upon these coating technologies since it is refreshed between central venous catheter accesses, providing a fresh reservoir of active ingredient periodically throughout the lifetime of the CVC device.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. An aqueous catheter lock solution comprising liposomes, wherein the liposomes encapsulate a nitric oxide releasing precursor material.

2. The catheter lock solution according to claim 1, wherein the liposomes have an outer membrane comprising a lipid material and cholesterol.

3. The catheter lock solution according to claim 2, wherein the lipid material and cholesterol are present in an approximately 1:1 molar ratio.

4. The catheter lock solution according to claim 2, wherein the lipid material comprises a phosphocholine material.

5. The catheter lock solution according to claim 4, wherein the phosphocholine material comprises dipalmitolylphosphatidylcholine (DPPC).

6. The catheter lock solution according to claim 1, wherein the liposomes have an outer membrane comprising a lipid material selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof.

7. The catheter lock solution according to claim 1, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

8. The catheter lock solution according to claim 1, wherein the nitric oxide releasing precursor material comprises N-diazeniumdiolate-modified spermine (SPER/NO).

9. The catheter lock solution according to claim 1, wherein the liposomes have an outer membrane comprising a lipid material and cholesterol, wherein the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

10. The catheter lock solution according to claim 9, wherein the lipid material and cholesterol are present in an approximately 1:1 molar ratio, the lipid material comprises dipalmitolylphosphatidylcholine (DPPC), and the nitric oxide releasing precursor material comprises N-diazeniumdiolate-modified spermine (SPER/NO).

11. The catheter lock solution according to claim 1, wherein the nitric oxide releasing precursor material has a nitric oxide releasing capacity, and wherein the nitric oxide releasing precursor retains at least 80% of its nitric oxide releasing capacity after three months of storage.

12. The catheter lock solution according to claim 1, wherein the nitric oxide releasing precursor material is dissolved in an aqueous solution having a basic pH in the range from about 7.1 to about 13.

13. A method for preparing the catheter lock solution according to claim 1, comprising:
obtaining a quantity of lyophilized liposomes which encapsulate a nitric oxide releasing precursor material; and
rehydrating the lyophilized liposomes to form the catheter lock solution.

14. The method according to claim 13, wherein the lyophilized liposomes have an outer membrane comprising a lipid material and cholesterol, wherein the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

15. A kit for preparing the catheter lock solution according to claim 1, comprising:
a quantity of lyophilized liposomes which encapsulate a nitric oxide releasing precursor material; and
a quantity of an aqueous rehydrating solution.

16. The kit according to claim 15, wherein the lyophilized liposomes have an outer membrane comprising a lipid material and cholesterol, wherein the lipid material is selected from dipalmitoylphosphatidylcholine (DPPC), dimyristoylphosphatidylcholine (DMPC), distearoylphosphatidylcholine (DSPC), dipalmitoyltrimethylammoniumpropane (DPTAP), dipalmitoylphosphatidylglycerol (DPPG), and mixtures thereof, wherein the nitric oxide releasing precursor material is selected from N-diazeniumdiolate-modified dipropylenetriamine (DPTA/NO), N-diazeniumdiolate-modified diethylamine (DEA/NO), N-diazeniumdiolate-modified diethylenetriamine (DETA/NO), N-diazeniumdiolate-modified N-propyl-1,3-propanediamine (PAPA/NO), N-diazeniumdiolate-modified L-proline (PROLI/NO), S-nitrosothiol (RSNO), S-nitrosoglutathione (GSNO), S-nitroso-N-acetyl penicillamine (SNAP), N-diazeniumdiolate-modified spermine (SPER/NO), and mixtures thereof.

17. A method of preparing liposomes which encapsulate a nitric oxide releasing precursor material, comprising:
combining a lipid material and cholesterol in an organic solvent for form an organic phase, wherein the lipid material and cholesterol are present in an approximately 1:1 molar ratio;
adding an aqueous solution comprising a nitric oxide releasing precursor material to the organic phase; and
evaporating the organic solvent, causing liposomes to form and encapsulate the nitric oxide releasing precursor material.

18. The method of preparing liposomes according to claim 17, wherein the organic solvent is selected from chloroform, methanol, diethyl ether, isopropyl ether, cyclohexane, ethanol, and mixtures thereof.

19. The method of preparing liposomes according to claim 17, wherein the lipid material comprises dipalmitolylphosphatidylcholine (DPPC) and the nitric oxide releasing precursor material comprises N-diazeniumdiolate-modified spermine (SPER/NO).

20. The method of preparing liposomes according to claim 17, wherein the aqueous solution has a basic pH in the range from about 7.1 to about 13.

21. The method of preparing liposomes according to claim 20, wherein the aqueous solution pH is adjusted with sodium hydroxide.

22. The method of preparing liposomes according to claim 17, further comprising the step of lyophilizing the liposomes.

23. The method of preparing liposomes according to claim 22, wherein the nitric oxide releasing precursor material has a nitric oxide releasing capacity, and wherein the nitric oxide releasing precursor retains at least 80% of its nitric oxide releasing capacity after three months of storage.
